(19) United States
(12) Patent Application Publication
(10) Pub. No.: US 2008/0230622 A1
(43) Pub. Date: Sep. 25, 2008

(54) Disseminating Device for Volatile Liquid

(76) Inventors: Colin Brown, Bracknell Berkshire (GB); Guy Edward Naish, Bicester Oxfordshire (GB); Kishen Gohil, New Malden Surrey (GB)

Correspondence Address: CURATOLO SIDOTI CO., LPA, 24500 CENTER RIDGE ROAD, SUITE 280, CLEVELAND, OH, 44145, US

(21) Appl. No.: 10/569,885

(22) PCT Filed: Aug. 20, 2004
(86) PCT No.: PCT/CH2004/000529

§ 371 Date: Apr. 16, 2008

(30) Foreign Application Priority Data
Sep. 2, 2003 (GB) 0320510.1

(51) Int. Cl. A61L 9/12; A61L 9/04

(52) U.S. Cl. 239/6; 239/44

(57) ABSTRACT

An apparatus adapted to disseminate volatile liquid, such as a fragrance, into an atmosphere comprises a reservoir (1) containing volatile liquid and, extending into and therefrom, an essentially cylindrical liquid transfer member (4) that transfers liquid from the reservoir to an evaporating surface (6) through which the transfer member passes by means of a hole in the evaporating surface. The evaporating surface comprises a rigid sheet that extends essentially laterally from the transfer member and bears on its surface capillary channels adapted to accept liquid from the transfer member and spread it over the surface of the evaporating surface. The transfer member is elastically compressible in diameter, with a diameter in its non-compressed form greater than that of the hole, and, prior to putting into service of the apparatus, is held in a compressed form of diameter smaller than that of the hole, such that, when the compression is released, the transfer member expands into liquid transfer contact with the evaporating surface. This ensures optimal liquid transfer contact between transfer member and evaporating surface.

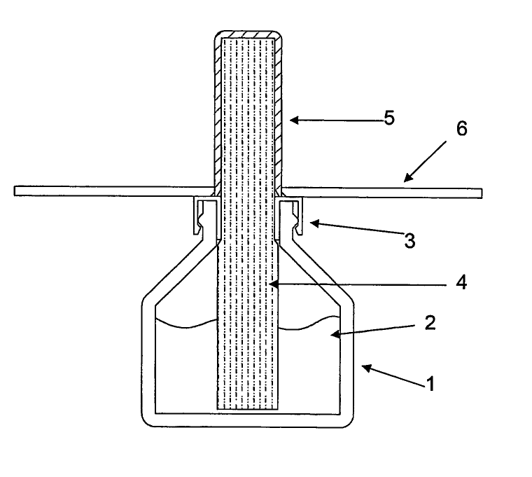

This invention relates to apparatus for disseminating volatile liquids into an atmosphere.

One very common apparatus for disseminating a volatile liquid, such as a fragrance, an insecticide, a fungicide, a medicament, etc., into an atmosphere consists of a porous transfer member, such as a fibrous wick, that is in contact with a reservoir of volatile liquid. Liquid rises up this wick and evaporates into the atmosphere. This system has drawbacks, such as the low surface area for evaporation. It has been proposed to overcome this disadvantage by fitting to such a transfer member an external capillary sheet, that is, a sheet extending essentially perpendicularly from the transfer member and that comprises channels of capillary dimensions, to which volatile liquid can pass and travel along for evaporation. This sheet generally contacts the transfer member by means of a hole in the sheet through which the transfer member protrudes and within which it fits snugly, at least some of these channels contacting the transfer member such that liquid can transfer from the member to the sheet (“liquid transfer contact”).

One of the problems encountered with such an apparatus is securing the necessary liquid transfer contact. If, to allow easy fitting, the hole in the sheet is too big with respect to the wick diameter, even if only slightly so, the transfer of liquid will be diminished, and may not even take place at all. On the other hand, if the hole is too small and the sheet has to be forced on to the wick, the wick may be damaged. In addition, the desired good liquid transfer may not still take place, because that part of the sheet surrounding the hole may be deformed slightly in the direction of the top of the wick, thus reducing liquid transfer contact or even eliminating it completely. Moreover, the precision engineering that would be necessary to make a wick and sheet that matched with the necessary exactness would result in an unacceptably high price for a mass market production article, especially as fibrous wicks are extremely difficult to engineer accurately.

It has now been found that these problems can readily be overcome by a new construction. The invention therefore provides an apparatus adapted to disseminate volatile liquid into an atmosphere, the apparatus comprising a reservoir containing volatile liquid and, extending into and therefrom, an essentially cylindrical liquid transfer member that transfers liquid from the reservoir to an evaporating surface through which the transfer member passes by means of a hole in the evaporating surface, the evaporating surface comprising a rigid sheet that extends essentially laterally from the transfer member and that bears on its surface capillary channels adapted to accept liquid from the transfer member and spread it over the surface of the evaporating surface, the transfer member being elastically compressible in diameter, with a diameter in its non-compressed form greater than that of the hole, and, prior to putting into service of the apparatus, being held in a compressed form of diameter smaller than that of the hole, and, on release of the compression, expandable into liquid transfer contact with the evaporating surface.

The invention additionally provides a method of ensuring optimum liquid transfer contact between a transfer member adapted to transfer volatile liquid to be disseminated into an atmosphere and a capillary channel-bearing evaporating surface that makes liquid transfer contact with the transfer member by means of a hole through which the transfer member protrudes, comprising supplying the transfer member in compressed form, such that its compressed diameter is smaller than that of the hole but its uncompressed diameter is at least equal to that of the hole, and, in putting the apparatus into service, of releasing the compression.

The reservoir and the evaporating surface may be any suitable reservoir or any suitable evaporating surface made of any suitable material. Reservoirs are well known to the art and suitable evaporating surfaces are described in, for example, co-pending International patent application No. PCT/CH 2004/000102.

The liquid transfer member may be any suitable member, with the proviso that it must be capable of being elastically compressed to a diameter smaller than its uncompressed diameter, such that

(a) the compressed diameter is sufficiently small such that the external diameter of the sleeve matches that of the hole in the evaporating surface; and

(b) the uncompressed diameter is greater than that of the hole in the evaporating sheet.

Provided that the liquid transfer member meets the compressibility criteria specified hereinabove, the material from which the transfer member is made may be any suitable material. Although it is advantageously any of the materials already known and used in the manufacture of air freshener wicks and the like, from the point of view of availability, reliability and cheapness, it is not restricted to these, and the skilled person will readily be able to select a suitable material for any given application. Typical examples of suitable transfer member material include natural fibres such as cellulose and synthetic fibres such as polyester, nylon and polypropylene. A typical example of a polyester fibrous wick contained within a non-woven outer sheath is the Sorbarod range of products from Baumgartner Fibertec SA, Crisser-Lausanne, Switzerland.

The means by which the transfer member is compressed and held in a compressed state may be any suitable means. For example, the compression retention means for the transfer member may be a cylindrical member, typically a sleeve or cap of material sufficiently rigid to resist the pressure of the transfer member seeking to return to normal size. This is typically a plastics material, but other materials such as ceramics or metals may be used, if desired. A cap is a preferred member, as it prevents leakage or evaporation. The compression of the transfer member may be achieved by any convenient means, for example, by extruding the transfer member in compressed form into the retention means.

The compression retention means described hereinabove may be held in place by any suitable means prior to its removal when the apparatus is put into operation. For example, if it is a cap, it may have a screw fitting that cooperates with a corresponding screw fitting on the neck of the reservoir. In another embodiment, it may be part of the closure of the reservoir and able to be broken off when operation of the apparatus is desired, for example, by making weak part of the cap, so that it will break at that point.

The evaporating surface is a capillary sheet, that is, a sheet of rigid material that comprises in at least one of its surfaces capillary channels. At least some of these channels are in liquid transfer contact

The apparatus according to the invention may be supplied in one part, or it may be supplied in two parts, evaporating surface and reservoir/transfer member/compression retention means. To put such an apparatus into operation, the evaporating surface is put over the sleeve, and the sleeve is then removed. The evaporating member is held in such a position that the subsequent expansion of the transfer member will bring it into liquid transfer contact with the evaporating surface. Any suitable means may be used here, for example, a shoulder built into a cap that both covers the transfer member and acts as the sleeve. This cap can be designed to snap off at an appropriate place by its being twisted, allowing the transfer member to expand.

The invention is further described with reference to the drawing, which depict a preferred embodiment and is not intended to be limiting in any way on the scope of the invention.

A reservoir 1 contains a volatile liquid (2), to be disseminated into an atmosphere. The reservoir has the form of a bottle with a neck, into which neck fits an insert 3. The insert has a central orifice, through which tightly fits a porous wick 4. The wick extends from near the bottom of the reservoir through the insert 3 into the atmosphere, thus providing the only means for the liquid leave the reservoir and reach the atmosphere. That part of the wick 4 protruding from the insert 3 is completely covered by a cap 5 that extends down to the insert 3. This part of the wick is compressed from its natural size and is held at reduced size by the cap 5. Around the cap 5 is fitted a capillary sheet 6, by means of an orifice in the sheet whose diameter matches the outer diameter of the cap 5, but which diameter is smaller than the normal diameter of the wick 4.

When the cap 5 is removed, the wick 4 expands to its normal diameter. In doing this, it makes tight contact with the orifice in the capillary sheet 6. This contact holds the capillary sheet 6 tightly in place and allows the passage of liquid from the wick 4 to the capillary sheet 6 for evaporation into the atmosphere.

1. An apparatus adapted to disseminate volatile liquid into an atmosphere, the apparatus comprising a reservoir containing volatile liquid and, extending into and therefrom, an essentially cylindrical liquid transfer member that transfers liquid from the reservoir to an evaporating surface through which the transfer member passes by means of a hole in the evaporating surface, the evaporating surface comprising a rigid sheet that extends essentially laterally from the transfer member and that bears on its surface capillary channels adapted to accept liquid from the transfer member and spread it over the surface of the evaporating surface, the transfer member being elastically compressible in diameter, with a diameter in its non-compressed form greater than that of the hole, and, prior to putting into service of the apparatus, being held in a compressed form of diameter smaller than that of the hole, and, on release of the compression, expandable into liquid transfer contact with the evaporating surface.

2. An apparatus according to claim 1, in which the transfer member is held in compressed form by a rigid cylindrical member.

3. An apparatus according to claim 2, in which the cylindrical member has an external diameter that is the same as the diameter of the hole.

4. An apparatus according to claim 2, in which the apparatus is a single unit.

5. An apparatus according to claim 2, in which the apparatus consists of two units, with a separate evaporating surface that is adapted to be fitted over a cylindrical member prior to the removal of the cylindrical member.

6. A method of ensuring optimum liquid transfer contact between a transfer member adapted to transfer volatile liquid to be disseminated into an atmosphere and a capillary channel-bearing evaporating surface that makes liquid transfer contact with the transfer member by means of a hole through which the transfer member protrudes, comprising supplying the transfer member in compressed form, such that its compressed diameter is smaller than that of the hole but its uncompressed diameter is at least equal to that of the hole, and, in putting the apparatus into service, of releasing the compression.
